(19) United States
(12) Patent Application Publication
(10) Pub. No.: US 2016/0303275 A1
(43) Pub. Date: Oct. 20, 2016

(54) LEUCO DYES WITH HOT MELT BINDING MATRIX

(71) Applicant: The Procter & Gamble Company, Cincinnati, OH (US)

(72) Inventors: Laveeta JOSEPH, Cncinnati, OH (US); Thomas James Klofta, Cincinnati, OH (US); Johannson Jimmy Tee, JR., Mason, OH (US); Italo Corzani, Chieti (IT)

(21) Appl. No.: 15/093,791

(22) Filed: Apr. 8, 2016

Related U.S. Application Data
(60) Provisional application No. 62/147,258, filed on Apr. 14, 2015.
(60) Provisional application No. 62/293,899, filed on Feb. 11, 2016.

(51) Int. Cl. A61L 15/56; A61F 13/42; A61L 15/20

(52) U.S. Cl. CPC A61L 15/56; A61L 15/20; A61F 13/42; A61F 2013/422; A61F 2013/427

(57) ABSTRACT

A wetness indicator composition comprising a leuco dye, a color developer, and a hot melt binding matrix.

### FIELD OF INVENTION

Disclosed are leuco dye formulations in a hot melt binding matrix, for use as wetness/fluid indicators in absorbent articles.

### BACKGROUND OF THE INVENTION

Many disposable absorbent articles comprise a wetness indicator. Wetness indicator compositions may comprise a colorant adapted to change in appearance, i.e., appear, disappear, change color, etc., upon contact with liquids such as urine, runny bowel movements, menses, etc., in the article. The color changing active used in many wetness indicator compositions are pH indicators. However, current pH-based wetness indicators may be unreliable, having issues such as premature triggering and/or leaching, plus there are limits as to the variety of beginning and final color options. Therefore, there is a continuing need for simple wetness/fluid indicators that can provide a variety of color options and a continuing need for ways to incorporate such wetness/fluid indicators into absorbent articles.

### SUMMARY OF THE INVENTION

A wetness indicator formulation is provided, comprising a leuco dye, a color developer, and a hot melt binding matrix.

### BRIEF DESCRIPTION OF THE DRAWINGS

FIG. 1 is a top view of an absorbent article according to an aspect of the invention.

### DETAILED DESCRIPTION OF THE INVENTION

### Definitions

“Article for baby care or feminine care” means products and/or methods relating to disposable absorbent and/or non-absorbent articles including adult incontinence garments, bibs, diapers, training pants, infant and toddler care wipes; catamenial pads, incontinence pads, interlabial pads, panty liners, pessaries, sanitary napkins, tampons and tampon applicators, and/or wipes.

As used herein, the term “colorant” refers to any dye, ink, pigment, inks that comprise dyes or pigments, pH indicators, metal indicators, oxidation or reduction indicators, solvatochromic colorants, biological colorant indicators that change color upon contact with a biological component of an exudates, or any material that has the effect of changing its color or the color of its environment, or any combination thereof.

As used herein, the term “permanent colorant” refers to a colorant that maintains its color independent of environmental factors or one that does not change its color under any circumstance, such as a pH change or exposure to a liquid or specific components of the liquid, high humidities, or high or low temperatures. “Absorbent core” means a structure typically disposed between a topsheet and backsheet of an absorbent article for absorbing and containing liquid received by the absorbent article and may comprise one or more substrates, absorbent polymer material disposed on the one or more substrates, and a thermoplastic composition on the absorbent particulate polymer material and at least a portion of the one or more substrates for immobilizing the absorbent particulate polymer material on the one or more substrates.

“Comprise,” “comprising,” and “comprises” are open ended terms, each specifies the presence of what follows, e.g., a component, but does not preclude the presence of other features, e.g., elements, steps, components known in the art, or disclosed herein.

“Consisting essentially of” is used herein to limit the scope of subject matter, such as that in a claim, to the specified materials or steps and those that do not materially affect the basic and novel characteristics of the subject matter.

“Diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

A “nonwoven” is a manufactured sheet, web, or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm). “Pant” or “training pant”, as used herein, refer to disposable garments having a waist opening and leg openings designed for infant or adult wearers. A pant may be placed in position on the wearer by inserting the wearer's legs into the leg openings and sliding the pant into position about a wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened).

### Leuco Dyes

The wetness indicating compositions that are utilized in this invention comprise a leuco dye and a color developer formulated into a hot melt binding matrix. A leuco dye (from the Greek leukos=white) is a dye whose molecule can acquire two chemical forms, one of which is substantially colorless. Leuco dyes are often classified according to the different chemical mechanisms that causes the dye molecule to change from its substantially colorless (leuco) form to a colorful form or vice versa. Therefore in the literature, one can find leuco dyes classified in categories such as thermochromic leuco dyes, whose change from a colorless to a colorful state (or vice versa) is caused by a variation in temperature; photochromic leuco dyes whose change from a colorless to a colorful state (or vice versa) is caused by light; solvatochromic leuco dyes whose change from a colorless to a colorful state (or vice versa) is caused by the contact with certain solvents and in particularly with a solvent of a certain well defined polarity; biochromic leuco dyes whose change from a colorless to a colorful state (or vice versa) is caused by the contact with specific biological entities or components; redox leuco dyes whose change from a colorless to a colorful state (or vice versa) is caused by chemical oxidation or reduction; electrochromic leuco dyes whose change from a colorless to a colorful state (or vice versa) is caused by the passage of an electric current; ionochromic leuco dyes whose change from a colorless to a colorful state (or vice versa) is caused by the interaction with specific ions and/or by a change in their concentration; halochromic leuco dyes whose change from a colorless to a colorful state (or vice versa), according to the initial literal meaning of the word (from the Greek halos=salt), ie., is caused by the interaction with specific salts etc. In the current technical literature today, it is common to find the term “halochromic” often associated with dyes and for leuco dyes in particular that change from an initial color (a colorless state in the case of leuco dyes) to a different colorful state as a consequence of a change in the concentration of hydrogen ions. In this slightly different meaning, halochromic leuco dyes are therefore a sub-class of the more general ionochromic leuco dyes, where a change in concentration of hydrogen ions causes the leuco dye to change from a colorless to a colorful state, or vice versa.

Thus, a leuco dye is a dye whose molecules can acquire two forms, one of which is substantially colorless. Without being bound by theory, a color developer may function as a Bronsted or Lewis acid or a strong hydrogen bonding ingredient, causing the colorless leuco dye to change to a colored dye.

For example, one particular leuco dye is crystal violet lactone, hereinafter abbreviated as CVL and chemically named as 3,3-Bis(p-dimethylaminophenyl)-6-dimethylaminophthalide 6-(Dimethylamino)-3,3-bis[p-(dimethylamino)phenyl]phthalide, with a CAS# of 1552-42-7. CVL is colorless when its five membered lactone ring structure remains intact. When reacted with a suitable developer like certain Lewis acid electron acceptor molecules, the lactone ring typically breaks open, resulting in a highly conjugated CVL molecule with multiple resonance structures. This highly conjugated ring-opened CVL molecule now appears blue to the naked eye. The CVL ring-opening reaction occurs due to its dissociation of the C-0 bond ground state in the presence of a proton donor or electron pair acceptor or even strong hydrogen bonding agents. This ring-opening reaction results in the formation of the highly conjugated and ring-opened blue cation. This colorless to blue colored reaction makes CVL an attractive active colorant not only for use within wetness indicator compositions, but also as a photopolymerization initiator and as the main active colorant in thermochromic layered structures.

As noted, the CVL lactone ring opening results from the reaction with a suitable color developer molecule. Many developers have the ability to donate a proton(s) to the CVL or accept electrons from the CVL molecule. This acidity of the developer leads to the ring opening of the CVL that causes the color change from colorless to blue. Not to be bound by theory, but the colorless ring-closed form of CVL is also referred to as its spiropyran form where the spiro carbon is sp3 hybridized and the 5-membered lactone ring remains intact. This sp3 hybridization of the spiro carbon limits the conjugation of the CVL such that the ring-closed form of CVL remains colorless to the naked eye. But, upon exposure to a proton(s) donating molecule, like a Bronsted acid, or an electron accepting molecule, like a Lewis acid, the lactone ring of CVL opens as result of bond breakage of the oxygen to the Spiro carbon. With the ring opening, the spiro carbon now becomes sp2 hybridized and multiple resonance structures are possible in this highly conjugated molecule. With this enhanced resonance and conjugation of bonds in the ring-opened CVL structure, the CVL molecule becomes intensely blue in color.

Not only does proton donation lead to the CVL's ring opening, but molecules with strong hydrogen bonding capabilities and/or Lewis acid properties can also induce the CVL's lactone ring-opening reaction. In addition, certain cationic atoms like mercury and copper, are known to intensify the blue color by complexing with the ring opened CVL.

For a color developer that is a proton donor, the reaction is depicted as follows where the five membered lactone ring structure on the left is colorless and the protonated structure on the right is blue:

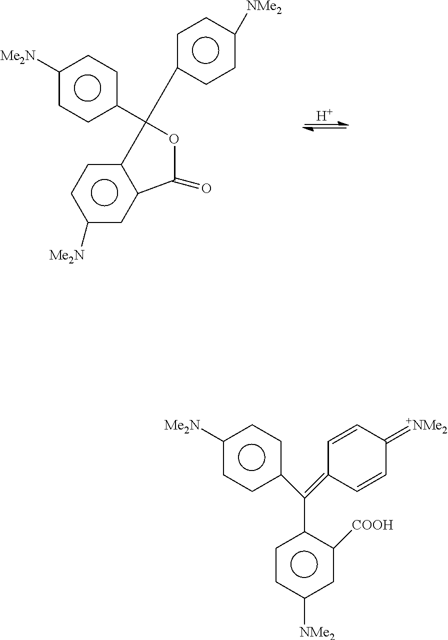

Crystal violet lactone is the phthalide based colorant (spirolactones) and the position of equilibrium is determined by pH rather than a redox process. As noted for crystal violet lactone, its structure is colorless when the spa center is formed and destroys the conjugation, and hence, the color of the chromophore. Other type of leuco dyes and colorants function by different mechanisms. For example, the colorless to colored change in the leucoquinone class of leuco colorants is activated by redox reactions. Another class of leuco colorants are light activated and these include leuco derivatives of the oxazine, thiazine, and phenazine dyes. Many of these are used in photoimaging applications.

Also other phthalide based leuco colorants, especially the ones having the same basic molecular structure of CVL may change from a colorless to a strongly colored state through a similar chemical mechanism and can be used in the present invention. Preferred leuco dyes of such type include heterocyclic analogues of CVL, in particular the ones with indole and pyridine rings, an example of which is 3-(1,2-Dimethyl-3-indolyl)-3-[4-(diethylamino)-2-methylphenyl]phthalide, identified also with the CAS number 36499-49-7 and also commercially known with the trade dye name of NC BLUE 3. Another leuco dye useful in these compositions include alpha-naphtholphthalein.

Other types of leuco dyes, in changing from a colorless to a colorful state, function by different chemical mechanisms. For example, the colorless to colored change and vice versa in the leuco-quinone class of leuco colorants is activated by redox reactions. Other classes of leuco colorants are light activated and these include leuco derivatives of the oxazine, thiazine, and phenazine dyes. Other leuco colorants may change from a colorless to a colored state when activated by the presence and concentration of certain specific ions and therefore by the presence of chemical compounds capable of releasing certain active ions like acids, salts etc. Such leuco colorant are known as ionochromic or halochromic. In a particular embodiment of the present invention these active ions are hydrogen ions, and said ionochromic/halochromic leuco dyes can therefore be used also as pH indicators. Examples of ionochromic leuco dyes of this type usable in the present invention, include e.g. thymolphthalein, alpha-naphtholphthalein, 4,5,6,7-tetrabromo-phenolphthalein, 3′,3″,5′,5″-tetrabromo-phenolphthalein, o-cresolphthalein, phenolphthalein, xylenolphthalein, guiacolphthalein, Ethyl bis (2,4-dinitrophenyl) acetate, bis-[9-(diethylamino)-5H-benzo[a]phenoxazin-5-iminium] sulfate (Nile Blue A), Quinoline Blue, Heptamethoxy Red, 3-nitrophenol, pinachrome.

Additional specific leuco dyes besides crystal violet lactone may include, for example, phthalide leuco dyes, triarylmethane leuco dyes, and fluoran leuco dyes. Examples may include (1) Triarylmethane-based dyes, e.g. 3,3-bis(p-dimethylaminophenyl)-6-dimethylaminophthalide, 3,3-bis (p-dimethylaminophenyl)phthalide, 3-(p-dimethylaminophenyl)-3-(1,2-dimethylindol-3-yl)phthalide, 3-(pdimethylaminophenyl)-3-(2-methylindol-3-yl)phthalide, 3,3-bis(1,2-dimethylindol-3-yl)-5-dimethylaminophthalide, 3,3-bis(1,2-dimethylindol-3-yl)-6-dimethylaminophthalide, 3,3-bis(9-ethylcarbazol-3-yl)-6-dimethylaminophthalide, 3,3-bis(2-phenylindol-3-yl)-6-dimethylaminophthalide, 3-pdimethylaminophenyl-3-(1-methylpyrrol-3-yl)-6-dimethylaminophthalide, etc. (2) Diphenylmethane-based dyes, e.g., 4,4′-bisdimethylaminobenZhydryl benzyl ether, N-halophenylleucoauramine, N-2,4,5-trichlorophenyl-leucoauramine, etc. (3) Lactam-based dyes, e.g., rhodamine-B-anilinolactam, rhodamine-(p-nitroanilino)lactam, rhodamine-(o-chloroanilino)lactam, etc. (4) Fluoran-based dyes, e.g., 3-dimethylamino-7-methoxyfluoran, 3-diethylamino-6-methoxy?uoran, 3-di-ethylamino-7-methoxyfluoran, 3-diethylamino-7-chloro fluoran, 3-diethylamino-6-methyl 7-chlorofluoran, 3-di-ethylamino-6,7-dimethylfluoran, 3-(N-ethyl-p-toluidino)-7-methylfluoran, 3-diethylamino-7-(N-acetyl-N-methylamino)fluoran, fluoran, 3-diethylamino-7(N-methylamino)fluoran, 3-diethylamino-7-dibenzylaminofluoran, 3-diethylamino-7-(N-methyl-N benzylamino)fluoran, 3-diethylamino-7-(N-chloroethyl-N-methylamino)fluoran, 3-diethylamino-7-N diethylaminofluoran, 3-(N-ethyl-p-toluidino)-6-methyl-7-phenylaminofluoran, 3-(N-ethyl-p-toluidino)-6-methyl-7-(p-toluidino) fluoran, 3-diethylamino-6-methyl-7-phenylamino fluoran, 3-dibutylamino-6-methyl-7-phenylaminofluoran, 3-diethylamino-7-(2 carbomethoxyphenylamino)fluoran, 3-(N-cyclohexyl-N-methylamino)-6-methyl-7-phenylaminofluoran, 3-pyrrolidino-6-methyl-7-phenylaminofluoran, 3-piperidino-6-methyl-7-phenylaminofluoran, 3-diethylamino-6-methyl-7-(2,4-dimethylamino)fluoran, 3-diethylamino-7-(o chlorophenylamino)fluoran, 3-dibutylamino-7-(o-chlorophenylamino)fluoran, 3-pyrrolidino-6-methyl-7-(p-butylphenylamino)fluoran, 3-(N-methyl-N-n-amylamino)6-methyl-7-phenylaminofluoran, 3-(N-ethyl-N-namylamino)-6-methyl-7-phenylaminofluoran, 3-(N-ethyl-N isoamylamino)-6-methyl-7-phenylaminofuluoran, 3-(N-methyl-N-n-hexylamino)-6-methyl-7-phenylaminofluoran, 3-(N-ethyl-N-n-hexylamino)-6-methyl-7-phenylaminofluoran, 3-(N-ethyl-N-[3-ethylhexylamino)-6-methyl-7-phenylaminofluoran, etc. The basic dyes useful in this invention are not limited to those exemplified above, and at least two of them can be used in admixture.

The wetness indicators of the present invention may comprise from about 0.01% to about 20.0% by weight of a leuco dye. The wetness indicator may comprise more than one leuco dye and may also comprise other colorants. Other colorants may be a dye, an ink, a pigment, a pH indicator, or combinations thereof. In addition, other colorants may be a permanent colorant/dye, an ink, a pigment like e.g. titanium dioxide or aluminum silicates or zinc oxide, a pH indicator, a redox indicator, or combinations thereof. In such instances, the total colorant in the wetness indicator may be from about 0.02% to about 30.0% by weight.

Some examples of oil soluble permanent colorants include D&C Yellow No. 11, D&C Red No. 17, D&C Red No. 21. D&C Red No. 27, D&C Violet No. 2, D&C Green No. 6, and D&C Orange No. 5. Additional permanent colorants include Pigment Red 146 (CAS#5280-68-2), Pigment Red 122 (CAS#980-26-7), Pigment Orange 16 (CAS#6505-28-8), red beet extract, and beta-carotene.

Some representative examples of liquid-activated colorants that can be used in the practice of this invention include: Malachite green, brilliant green, crystal violet, erythrosine B, methyl green, methyl violet 2D, picric acid, naphthol yellow S, quinaldine red, eosine Y, metanil yellow, m-cresol purple, thymol blue, xylenol blue, basis fuchsin, eosin B, 4-p-aminophenol(azo)benzenesulphonic acid-sodium salt, cresol red, m-cresol red, m-cresol purple, martius yellow, phloxine B, acid phloxine, methyl yellow, bromophenol blue, congo red, methyl orange, crystal violet lactone, ethyl bis(2,4-dinitrophenyl) acetate, bromochlorophenol blue (water soluble or free acid form), ethyl orange, flourocene WS, bromocresol green, chrysoidine, methyl red sodium salt, alizarine red S-H20, cochineal, chlorophenol red, bromocresol purple, 4-naphtha, alizarin, nitrazine yellow, bromothymol blue, brilliant yellow, neutral red, rosalic acid, phenol red, 3-nitro phenol, orange II, phenolphthalein, o-cresolphthalein, nile blue A, thymolphthalein, aniline blue WS, alizarine yellow GG, mordant orange, tropaolin O, orange G, acid fuchsin, thiazol yellow G, indigo carmine, cresol red, methyl red, p-nitrophenol, and alizarin yellow R. In certain instances, it is advantageous to use the free acid form, free base form, or salt form of the colorants, or mixtures thereof.

Additional water-soluble colorants may include FD&C Blue No. 1, FD&C Blue No. 2, FD&C Green No. 3, FD&C Red No. 40, FD&C Red No. 4, FD&C Yellow No. 5, FD&C Yellow No. 6, C.I. Food Blue 5, and C.I. Food Red 7, D&C Yellow No. 10, D&C Yellow No. 7, D&C Yellow No. 2, D&C Yellow No. 8, D&C Orange No. 4, D&C Red No. 22, D&C Red No. 28, D&C Red No. 33, D&C Green No. 8, D&C Green No. 5, D&C Brown No. 1, and any combination thereof. Preferably, the colorant is soluble within the wetness indicator composition, but, as noted in certain instances, the colorant can function as intended by homogeneously suspending or dispersing it within the wetness indicator composition.

Additional suitable fluid colorants include water soluble colorants like direct dyes, acid dyes, base dyes, and various solvent-soluble colorants. Dispersed or suspended pigment colorants can also be employed into these wetness indicator compositions (liquid-activated formulations). Examples include, but are not limited to, C.I. Acid Yellow 73, C.I. Solvent Yellow 94, C.I. Acid Yellow 74, C.I. Solvent Orange 32, C.I. Solvent Red 42, C.I. Acid Orange 11, C.I. Solvent Red 72, C.I. Pigment Orange 39, C.I. Solvent Orange 18, C.I. Acid Red 87, C.I. Solvent Red 43, C.I. Pigment Red 90:1, C.I. Solvent Red 44, C.I. Solvent Red 45, C.I. Solvent Orange 16, C.I. Acid Red 91, C.I. Acid Red 98, C.I. Acid Red 92, C.I. Solvent Red 48, C.I. Pigment Red 174, Pigment Red 146 (C.I. No. 12485, CAS#5280-68-2)), Pigment Red 122 (CAS#980-26-7), Pigment Red 112 (CAS#6535-46-2), Pigment Red 101 (CAS#1309-37-1), Pigment Orange 34 (CAS#15793-73-4, Pigment Orange 16 (CAS#6505-28-8), Pigment Green 7 (CAS#1328-53-6), Pigment Blue 15:2 (CAS#12239-87-1), Pigment Blue 15 (CAS#147-14-8), Pigment Black 7 (CAS#1333-86-4), Pigment Red 176, Pigment Red 200, Pigment Red 254, Pigment Red 48:1, Pigment Red 48:2, Pigment Red 48:3, Pigment Red 52, Pigment Red 52:1, Pigment Red 57:1, Pigment Red 63:1, Pigment Violet 19, Pigment Violet 23, Pigment Yellow 12, Pigment Yellow 13, Pigment Yellow 14, Pigment Yellow 17, Pigment Yellow 74, Pigment Yellow 83, C.I. Acid Red 95, C.I. Solvent Red 73, C.I. Pigment Red 191, C.I. Acid Red 51, C.I. Food Red 14, C.I. Pigment Red 172, C.I. Solvent Red 140, C.I. Acid Red 93, C.I. Solvent Red 47, C.I. Acid Red 94, C.I. Solvent Red 141, C.I. Mordant Violet 25, C.I. Solvent Orange 17, C.I. Solvent Red 46, D&C Red 27(C.I. 45410:1), D&C Orange 5(C.I. 45370:2), and combinations thereof. More preferred fluid colorants are selected from the group consisting of D&C Red 27, D&C Orange 5, and combinations thereof.

Additional suitable colorants may include bromopyrogallol red, bromoxylenol blue, methylene blue, monoazo dyes such as acid alizarin violet N, monoazo pyrazoline dyes (such as acid yellow 34), diazo dyes (such as acid black 24), anthraquinone dyes (such as acid black 48), amphoteric anthraquinone dyes (such as acid blue 45), triphenylmethane dyes (such as acid fuchsin), phthalein type dyes (such as o-cresolphthalein), xanthene dyes (such as 2′7′ dichlorofluorescein eosin B), heterocyclic acridine aromatics (such as acridine orange), diphenylmethane dyes (such as auramine O), triphenylmethane dyes (such as basic fuchsin), cationic thiazine dyes (azure C), cationic anthraquinone dyes such as basic blue 47, phthalocyanine type dyes (such as strazon orange G), anthraquinone type (such as alizarin), neutral complex dyes (such as azure A eosinate), terpene type dyes (such as trans-beta-carotene), as well as combinations including at least one of the foregoing dyes.

Examples of colorants further include, but are not limited to, organic dyes, inorganic pigments, colored macromolecules, colored nanoparticles and materials. Examples of dyes include acridine dyes, anthraquinone dyes, arylmethane dyes, azo dyes, nitro dyes, nitroso dyes, phthalocyanine dyes, quinone-imine dyes, Aazin dyes, Indophenol dyes, oxazin dyes, Oxazone dyes, Thiazole dyes, xanthene dyes, Fluorene dyes, fluorone dyes, rhodamine dyes and natural dyes like beta-carotene, annatto, cochineal, caramel color, red beet extract, beet pigments, riboflavin, anthocyanin, carotenoids, apocarotenal, and paprika. Also suitable are carmelizing ingredients used to darken the color. Examples of pigments include Cadmium pigments: cadmium yellow, cadmium red, cadmium green, cadmium orange; Carbon pigments: carbon black (including vine blac, lamp black), ivory black (bone char); Chromium pigments: chrome yellow and chrome green; Cobalt pigments: cobalt violet, cobalt blue, cerulean blue, aureolin (cobalt yellow); Copper pigments: Azurite, Han purple, Han blue, Egyptian blue, Malachite, Paris green, Phthalocyanine Blue BN, Phthalocyanine Green G, verdigris, viridian; Iron oxide pigments: sanguine, caput mortuum, oxide red, red ochre, Venetian red, Prussian blue; Clay earth pigments (iron oxides): yellow ochre, raw sienna, burnt sienna, raw umber, burnt umber; Lead pigments: lead white, cremnitz white, Naples yellow, red lead; Mercury pigments: vermilion; Titanium pigments: titanium yellow, titanium beige, titanium white like titanium dioxide, titanium black; Ultramarine pigments: ultramarine, ultramarine green shade; Zinc pigments: zinc white like zinc oxide, zinc ferrite. Other examples include alizarin, alizarin crimson, gamboge, cochineal red, rose madder, indigo, Indian yellow, Tyrian purple, organic quinacridone, magenta, phthalo green, phthalo blue, pigment red.

### Color Developers

The wetness indicators of the present invention may include a color developer. Developers that can react with a leuco dye to break open the lactone ring include phenols or aromatic amines, other colorants, hydrogen bonding agents, Bronsted acids like carboxylic acids or metal salts or Lewis acids, and mixtures thereof. Specific developers include gallic acid (CAS#149-91-7), propyl gallate (CAS#121-79-9), octyl gallate (CAS#1034-01-1), dodecyl (lauryl) gallate (CAS#1166-52-5), Zinc 3,5-bis(alpha-methylbenzyl) salicylate (CAS#53770-52-8), salicylic acid and its salts and esters, 3,5-Di-tert-butylsalicylic acid (CAS#19715-19-6) and appreviated as TBSA, Benzyl 4-hydroxybenzoate (acronym of PHBB with CAS#94-18-8), 4.4′-Dihydroxybenzophenone (CAS#611-99-4), 2,4′-dihydrohybenzophenone (CAS#131-56-6), 2,2′,4,4′-Tetrahydroxybenzophenone (CAS#131-55-5), 2,2-bis(phydroxyphenyl) propane (common name of Bisphenol A with a CAS# of 80-05-7), Bis(4-hydroxyphenyl)methane (common name of Bisphenol F with a CAS# of 620-92-8), 4-Hydroxyphenyl sulfone (common name of Bisphenol S with a CAS# of 80-09-1), bis-(3-allyl-4-hydroxyphenyl) sulfone (CAS#41481-66-7), 4-[4′-[(1′-methylethyloxy) phenyl]sulfonyl]phenol (CAS#191680-38-8), 4-hydroxyphenyl 4-isoprooxyphenylsulfone (also known as D-8 with a CAS# of 95235-30-6), Phenol, 4-[[4-(2-propen-1-yloxy)phenyl]sulfonyl] (also known as BPS-MAE with a CAS# of 97042-18-7), 4-Hydroxy-4′-benzyloxydiphenylsulfone (also known as BPS-MPE with a CAS# of 63134-33-8), Urea Urethane Compound with a CAS# of 321860-75-7, 4,4′-bis(N-carbamoyl-4-methylbenzenesulfonamide)diphenylmethane (abbreviated as BTUM with a CAS# of 151882-81-4), 2,4′-Bis(hydroxyphenyl)sulfone (CAS#5397-34-2), 4,4′-(1-Phenylethylidene)bisphenol (also known as Bisphenol AP with a CAS# of 1571-75-1), 2,2′-Bis(4-hydroxy-3-methylphenyl)propane (also known as Bisphenol C with a CAS# of 79-97-0), Methyl bis(4-hydroxyphenyl)acetate (abbreviated as MBHA with a CAS# of 5129-00-0), 4,4′-Isopropyllidenebis(2-phenylpheno) (known as BisOPP-A with a CAS# of 24038-68-4), 1,7-bis(4-hydroxyphenylthio)-3,5-dioxaheptane (CAS#93589-69-6). Bisphenol A, Bisphenol B, p-Dihydroxylbenzene, p-Methylphenol, m-Nitrobenzoic acid, m-Aminobenzoic acid, tannic acid, phenolpropenoic acid, propyl gallate (CASA #121-79-9), gallic acid, octyl gallate, butyl gallate, hexyl gallate, decyl gallate, dodecyl (lauryl) gallate, 1,2,3-triazoles, thioureas, calcium chloride, magnesium chloride decahydrate, phenolphthalein, cresol red, PERGAFAST 201 (Benzenesulfonamide, 4-methyl-N-[[[3[[(4-methylphenyl)sulfonyl]oxy]phenyl]amino] carbonyl]), gallic acid, BPS MAE (CAS 97042-18-7), D-90 (CAS 191680-83-8), a urea urethane compound (CAS 3217860-75-7), bisphenol AP (1571-75-1), MBHA (5129-00-0), Bisphenol S, Zinc 3,5-bis(alpha-methylbenzyl) salicylate (CAS 53770-52-8), 3,5-Di-tert-butylsalicylic acid (CAS 19715-19-6), aluminum trichloride, aluminum distearate monochloride, Zinc Salicylate (CAS #16283-53-0), 4.4′-Dihydroxybenzophenone (CAS #611-99-4), Benzyl 4-Hydroxybenzoate (CAS #94-18-8).

Further examples of suitable developers include, zinc chloride, phenol resins, 4-tert-butylphenol, (x-naphthol, [3-naphthol, 4-acetylphenol, 4-tert-octylphenol, 4,4′-sec-butylidenephenol, 4-phenylphenol, 4,4′-dihydroxydiphenylmethane, 4,4′-isopropylidene diphenol, hydroquinone, 4,4′cyclohexylidene diphenol, 4,4-dihydroxy diphenyl sulfide, 4,4′-thiobis(6-tert-butyl-3-methylphenol), 4,4′-dihydroxydiphenyl sulfone, hydroquinone monobenzyl ether, 4-hydroxybenzophenone, 2,4-dihydroxybenzophenone, 2,4,4′trihydroxybenzophenone, 2,2′,4,4′tetrahydroxybenzophenone, dimethyl 4-hydroxyphthalate, methyl 4-hydroxybenzoate, ethyl 4-hydroxybenzoate, propyl 4-hydroxybenzoate, sec-butyl 4-hydroxybenzoate, pentyl 4-hydroxybenzoate, phenyl 4-hydroxybenzoate, benzyl 4-hydroxybenzoate, tolyl 4-hydroxybenzoate, chlorophenyl 4-hydroxybenzoate, phenylpropyl 4-hydroxybenzoate, phenyl ethyl 4-hydroxybenzoate, p-chlorobenzyl 4-hydroxybenzoate, p-methoxybenzyl 4-hydroxybenzoate, benzoic acid and its salts, other benzoic acid esters and their salts, phenol polymers and like phenol compounds.

Further examples of suitable developers include also inorganic compounds like silica and natural or synthetic silicates like clays, zeolites and the like, as well inorganic and organic compounds of polyvalent metals like oxides, halides, carbonates, sulfates, nitrates, acetates, formates, oxalates, benzoates, acetylacetonates, stearates, salicylates, hydroxides like sodium hydroxide and potassium hydroxide, and hydroxides of magnesium, aluminum, calcium, titanium, zinc, cadmium, nickel, cobalt, iron, manganese, copper, vanadium etc. like magnesium chloride, calcium chloride, zinc chloride, zinc bromide, zinc iodide, zinc oxide, zinc stearate, zinc glicynate, zinc resinates, aluminum trichloride, aluminum oxide, aluminum stearate, aluminum glycinate, aluminum acetylacetonate etc. silica and silicates as well as compounds of zinc, aluminum, copper, and magnesium are preferred; and, among metallic compounds, zinc compounds are the most preferred. Surprisingly, it has been found that zinc compounds, like e.g. zinc oxide or zinc salicylates, when used alone or together with another non-zinc containing developer, significantly strengthen or amplify the color to which leuco dyes turn upon contact with water and by reaction with the color developer(s).

It has also been surprisingly found that, especially when the used leuco dye is crystal violet lactone (CVL) or one of its structurally similar analogues, it is preferable that the color developer or developers used in the present invention have a low solubility in water, for example equal or lower than 10 grams per liter at 20° C. and more preferably equal or lower than 5 grams per liter at 20° C. This helps in avoiding possible premature changes of color before actual contact with a substantial amount of liquid water, e.g. for possible absorption of humidity from moist air. Also, this aids in keeping the developer locked into the wetness indicator matrix rather than leaching into the urine and ultimately diffusing to other regions of the diaper. It is optimum to keep the developer and leuco dye locked into the wetness indicator matrix so they can complex with one another to cause the color change.

In a particular embodiment of the present invention, in the case that one or more ionohromic/halochromic leuco dyes are used, the color developer(s) may be any chemical compound or blend of compounds able to release, upon addition of water or of water containing physiological fluids, the active ions that activate the leuco dye for changing from an initial colorless state to a final colorful state. In a more particular embodiment, when such active ions are hydrogen ions, any chemical compound having in contact with water a distinct basic or acidic reaction, depending on the fact that the used leuco dye changes from its colorless to its colorful state in a basic or in an acidic environment, may work as developers. Examples of suitable developers for hydrogen ions activated ionochromic leuco dyes that are activated for color change in an acidic environment, are typical inorganic or organic acids like e.g., as non limiting examples, phosphoric acid, citric acid, tartaric acid, sulfamic acid, benzensulfonic acid, stearic acid, isostearic acid, abietic acid, acidic rosins etc; as well as salts that in water have an acidic behavior like aluminum sulfate and its double sulfates (alums), hydrogen sulfates of sodium and potassium, ammonium halides etc. For ionochromic leuco dyes that are activated in a basic environment suitable developers may be for example inorganic or organic compounds and salts that in water have a distinct basic behavior, like e.g. hydroxides, carbonates and hydrogen carbonates of alkali and earth-alkali metals, stearates and oleates of sodium and potassium, sodium tetraborate, tri-sodium phosphate, guanidine carbonate, amines etc.

The wetness indicator compositions of the present invention comprise from about 0.1% to about 50.0% by weight of a color developer or of a blend of color developers, preferably from about 1.0% to about 30.0%.

### Hot Melt Binding Matrix

The wetness indicating compositions that are utilized in this invention comprise a hot melt binding matrix. Processing a hot melt binding matrix involves melting the components together at an elevated temperature, typically from at least about 50° C. to about 170° C., in some embodiments, from about 60° C. to about 130° C., in some embodiments from about 80° C. to about 120° C. In order to be hot melt processable, the wetness indicator composition must be heated to a temperature high enough so as to insure the adhesive flows readily but not so hot so as to cause degradation at an unacceptable rate. Thus, it is common to add an anti-oxidant and other stabilizers, e.g. UV stabilizers and UV absorbers, to the hot melt compositions in order to slow down the decomposition rate. An effective anti-oxidant to add is Irganox 1010 from BASF Incorporated. It may be difficult to achieve compatibility and stability of such wetness indicating components if processed at room temperature. It may also be difficult under some printing processes to print such compositions onto a substrate. But the present invention's components are melted together at elevated temperatures, and the hot melt liquid is applied and adhered to a substrate while at an elevated temperature to keep the composition in liquid molten state.

The hot melt binding matrix may comprise binding agents that can be any material that immobilizes the colorant, or combination of colorants, within the matrix to hinder leaching of the colorant(s) into a diaper core or other regions of an absorbent article. To optimize the contrast and vibrancy of the colors, it is much preferred to “lock” the colorant within the matrix before and after contact with a fluid like urine. The binding agents can not only hinder the leaching of the color outside of the matrix, but also aid in binding the entire wetness indicator composition to a component of the absorbent article. For example, the binder can aid in forming a strong bond between the surface of the diaper backsheet and the wetness indicator composition.

There are various materials which may be suitable for use as a binding agent in a hot melt binding matrix for the wetness indicators of the present invention. A number of different polymers and blends of polymers used in hot melt adhesives may be used as the primary binding agent to combine and mix the leuco dye with the color developer and other optional ingredients such as stabilizers, tackifiers, waxes, surfactants, viscosity modifiers, fillers, anti-oxidants and other colorants.

Such hot melt polymers, copolymers, terpolymers, and other materials include ethylene vinyl acetates (EVA), polyolefins like low density polyethylene (LDPE) and high density polyethylene (HDPE), the styrene-isoprene-styrene block tri- and co-polymers as trademarked as Kratons by Kraton Incorporated where a specific trademarked example is Kraton D-1113, atactic polypropylene and polypropylene homopolymers, oxidized polyethylene like Honeywell's A-C 6702 and A-C 330 and Henkel's Technomelt (REGISTERED™ symbol) line of polyolefins. Polyamides like Henkel's Macromelt (REGISTERED™ symbol here) 6072. Other hot melt components like polymethyl methacrylate, ethylene vinyl acetates (EVA) like Dupont Elvax (trademark symbol) line of EVA's, polymethacrylic acid, polyacrylic acid, ethylene-acrylic acid polymers (EAA) like Honeywell's A-C 5120, fully and partially neutralized salts of the ethylene-acrylic acid copolymers, ethylene-ethyl acetate, polyacrylates, ethylene-vinyl acetate copolymers and oxidized ethylene-vinyl acetate copolymers like Honeywell's A-C 645P, ethylene maleic anhydride copolymers, propylene maleic anhydride copolymers, polyethylene imines (PEI) like BASF's Lupasol (registered trademark symbol), polyurethanes, polyacryl amides, branched copolymers comprising monomeric units derived from acrylic acid and/or quaternary ammonium compounds and/or acrylamide, branched copolymers comprising one or more monomeric units derived from quaternary ammonium compounds, amine compounds, acrylamide compounds, acrylic acid compounds and mixtures thereof at various weight ratios within the polymer, another example is a copolymer of acrylamide reacted with one or more other nonionic monomers, for example non-acrylamide monomers, such as hydroxyalkylacrylate, for example hydroxypropylacrylate, another example is a branched copolymer of acrylamide reacted with bismethyleneacrylamide, a crosslinking agent, that converts a typical linear polyacrylamide into a branched polymeric structure, another copolymer example includes the reaction between a nonionic monomeric unit derived from an acrylamide compound and an anionic monomeric unit derived from acrylic acid or other suitable monomers that could become anionic where examples include anionic monomers selected from the group consisting of: monomers having at least one carboxylic function, for instance α,β-ethylenically unsaturated carboxylic acids or the corresponding anhydrides, such as acrylic, methacrylic or maleic acids or anhydrides, fumaric acid, itaconic acid, N-methacroylalanine, N-acryloylglycine, and their water-soluble salts, monomers that are precursors of carboxylate functions, such as tert-butyl acrylate, which, after polymerization, give rise to carboxylic functions by hydrolysis, monomers having at least one sulfate or sulfonate function, such as 2-sulfooxyethyl methacrylate, vinylbenzene sulfonic acid, allyl sulfonic acid, 2-acrylamido-2-methylpropane sulfonic acid (AMPS), sulfoethyl acrylate or methacrylate, sulfopropyl acrylate or methacrylate, and their water-soluble salts, monomers having at least one phosphonate or phosphate function, such as vinylphosphonic acid, etc., the esters of ethylenically unsaturated phosphates, such as the phosphates derived from hydroxyethyl methacrylate (Empicryl 6835 from Rhodia) and those derived from polyoxyalkylene methacrylates, and their water-soluble salts, and 2-carboxyethyl acrylate (CEA). Not to be bound by theory, but the inclusion of potential anionic moieties within the polymer backbone can aid in decreasing the leaching of a blue cationic form of crystal violet lactone by forming complex between the polymeric anionic and cationic colorant. The hot melt may also comprise polymers with a cationic monomeric unit, such as a cationic monomeric unit derived from cationic monomers selected from the group consisting of: N,N-(dialkylamino-ω-alkyl)amides of α,β-monoethylenically unsaturated carboxylic acids, such as N,N-dimethylaminomethylacrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride or methyl sulfate, and acryloyloxyethyltrimethylammonium chloride; 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT) and 2-hydroxy-N¹-(3-(2((3-methacrylamidopropyl)dimethylamino)-acetamido)propyl)-N¹,N¹,N³,N³,N³-pentamethylpropane-1,3-diaminium chloride (TRIQUAT), and. In one example, the cationic monomeric unit comprises a quaternary ammonium monomeric unit, for example a monoquaternary ammonium monomeric unit, a diquaternary ammonium monomeric unit and a triquaternary monomeric unit. In one example, the cationic monomeric unit is derived from MAPTAC. In another example, the cationic monomeric unit is derived from DADMAC. In still another example, the cationic monomeric unit is derived from 2-hydroxy-N¹-(3-(2((3-methacrylamidopropyl)dimethylamino)-acetamido)propyl)-N¹,N¹,N³,N³,N³-pentamethylpropane-1,3-diaminium chloride. Other polymers that can make up the hot melt includepolyamines, polypryrroles, polyimidazoles, polycarbonates, polyesters, styrene block copolymers, PVP, polyvacrylamide, polyacryldextran, polyalkyl cyanoacrylate, cellulose acetate, cellulose acetate butyrate, cellulose nitrate, methyl cellulose and other cellulose derivates, chitosan and chitosan derivatives, chitin and chitin derivatives, nylon 6,10, nylon 6,6, nylon 6, polyterephthalamide and other polyamides, polycaprolactones, polydimethylsiloxanes and other siloxanes, silicone rubbers, aliphatic and aromatic polyesters, polyethylene oxide, polyethylene-vinyl acetate, polyglycolic acid, polylactic acid and copolymers, poly(methyl vinyl ether/maleic anhydride), polystyrene, polyvinyl acetate phthalate, polyvinyl alcohol) polyvinylpyrollidone, copolymers of vinyl pyrrolidone and vinyl acetate like the PVP-VA S-630 copolymer from Ashland Chemical Inc., shellac, starch and modified starches, chitosans, fatty alcohols, primary alcohols of long carbon chain lengths of C24 to C50, ethoxylated fatty alcohols, ethoxylated primary alcohols of chainlengths of C24 to C50, fatty acids, and waxes such as paraffinic and microcrystalline, synthetic waxes like polyethylene waxes, natural waxes like beeswax, carnauba wax and mixtures thereof.

In some embodiments, the binding agent may be a hot melt adhesive, in some embodiments, a solvent-based binding matrix. Additional components of a hot melt adhesive binding matrix may include base polymers, tackifiers, waxes, rubbers, solvents, wetting agents, and/or anti-oxidants. Examples of base polymers used in hot melt adhesives may include ethylene-vinyl acetate (EVA) copolymers like those of the Elvax brand name and marketed by DuPont Incorporated; styrenic block copolymers like those from Kraton Incorporated, ethylene/acrylic acid copolymers like the AC brand marketed by Honeywell Incorporated, vinyl pyrrolidone/vinyl acetate copolymers, pyrrolindone homopolymers like those marketed by BASF Incorporated and marketed under the trade name of Luviskol, vinyl pyrrolidone homopolymers, polyamides; kraton polymers, ethylene/acrylic acid co-polymers, ethylene-acrylate copolymers; ethylene-vinylacetate-maleic anhydride terpolymer; ethylene-acrylate-maleic anhydride terpolymer; high molecular weight and water soluble polyethylene oxide polymers like the trademarked POLYOX resins from DOW Chemical Incorporated and include DOW's Polyox WSR N-10 polymeric resin, polyolefins such as low density and high density polyethylene, atactic polypropylene, oxidized polyethylene, polybutene-1; amorphous polyolefins like amorphous atactic propylene (APP), amorphous propylene/ethylene (APE), amorphous propylene/butane (APB), amorphous propylene/hexane (APH), and amorphous propylene/ethylene/butane; polyamides; styrene block copolymers (SBC); styrene/acrylic polymers and modified styrene/acrylic polymers; polycarbonates; silicone rubbers; polypyrrole based polymers; thermoplastic elastomers like natural and synthetic polyisoprene, polybutadiene rubber, butyl rubber, chloroprene rubber, ethylene-propylene rubber, epichlorohydrin rubber, polyacrylic rubber, polyether block amides; polymers of acrylates, alkyd resins, amides, amino resins, ethylene co-terpolymer resins such as EVA, epoxy resins, fluoropolymers, hydrocarbon resins, phenols, polyesters, olefins, polyurethanes, silicones and functionalized silicones, polystyrene and polyvinyls.

The binding agent may be employed in compositions at levels which are effective at immobilizing and stabilizing the colorant in its first state, including from about 1% to about 90%, from about 10% to about 75%, and from about 20% to about 65%, by weight of the wetness indicator composition.

The binding matrix may comprise a first and second binding agent. The second binding agent may be any material which may immobilize the colorant when the colorant is in its final color state. This immobilization helps to bind the colorant within the wetness indicator composition to prevent it from leaching to other regions of the diaper such as the diaper core. It should be noted that similar to the first binding agent, the second binding agent can function not only to hinder the leaching of the colorant outside of the wetness indicator composition but can also aid in bonding the entire wetness indicator composition to the material of interest within the absorbent article. For example, the second binding agent may aid in bonding the wetness indicator composition to the backsheet of the diaper. There are various materials which may be suitable for use as an additional binding agent for the wetness indicators of the present invention. For example, the binding agent might be a cationic agent to complex with anionic colorants. Or, the binding agent could be an anionic agent to complex with a cationic colorant like the blue and ring opened form of crystal violet lactone. In one embodiment, a binding agent may be selected from, but are not limited to, the second binding agents disclosed in U.S. Pat. No. 6,904,865 to Klofta.

Tackifiers suitable for hot melt adhesives include, without being limited to, natural resins like copals like gum copal, dammars, mastic, and sandarac; rosins and their derivatives; hydrogenated rosins like Eastman Chemical Companies trademarked Foral AX and Foral AX-E rosins; terpenes and modified terpenes; aliphatic, cycloaliphatic, and aromatic resins like C5 aliphatic resins, C9 aromatic resins, and C5/C9 aromatic/aliphatic resins, hydrogenated hydrocarbon resins, polybutenes and polyisobutenes, and their mixtures.

Waxes suitable for hot melt adhesives include, without being limited to, mineral waxes like paraffin and microcrystalline waxes; polyethylene waxes; polyethylene glycol type waxes like those trademarked as the Carbowax brand from DOW Chemical Incorporated; oxidized polyethylene waxes; polymethylene waxes, the bisstearamides like N,N′-ethylene bisstearamide trademarked as Acrawax from Lonza Incorporated, highly branched polymer waxes like Vybar™ from Baker Hughes; fatty amide waxes; natural and synthetic waxes like beeswax, soywax, carnuba, ozokerite, ceresin; waxes derived from both the Fisher-Tropsch and Ziegler-Natta processes; water soluble waxes, polyalkylene wax, polyethylene wax, and silicone waxes.

Additional additives for adhesives and hot melt adhesives may include plasticizers, like glyceryl tribenzoate, alkyl benzoates and benzoate esters like Eastman Chemical Corporation's trademarked Benzoflex 9-88, glycols like diethylene glycol, C12-15 alkyl benzoate, C2-C22 alkyl benzoates where the alkyl group is straight or branched or mixtures thereof, alkyl citrates, phthalates, phthalate esters, paraffin oils, and polyisobutylene; UV stabilizers and UV absorbers like the Tinuvin series as marketed by BASF Incorporated and include Tinuvin 770 and Tinuvin 928 and Tinuvin-(384-2) and Tinuvin-123 and combinations thereof; biocides and antimicrobial preservatives; antioxidants, like BHT, phospites and phosphates; antistatic agents; rosins and their derivatives; pigment, particle and powder wetting agents like polyhydroxystearic acid, polyglyceryl-4 isostearate, hexyl laurate, esters like isopropyl myristate, propylene carbonate, isononyl isononanoate, glyceryl behenate/eicosadioate, trihydroxystearin, C12-15 alkyl benzoate, C2-C22 alkyl benzoates where the alkyl group is straight or branched or mixtures thereof, triethoxycaprylysilane, castor oil; and viscosity modifiers. The wetting agent can be a combination of an ester like isononyl isononanoate and a surfactant like polyhydroxystearic acid. Optionally, solvents like mineral oil, isoparaffins, alkanes like hexane, silicone fluids, esters, alcohols, polyethylene glycols, glycerin, glycols, and water can be added to reduce the viscosity of the composition or to increase the solubility of other ingredients or change other strategic properties of the wetness indicator composition.

The matrix, including both the first and second binding agents, may be employed in wetness indicator compositions at levels which are effective at immobilizing and stabilizing the colorant, including from about 5% to about 95%, from about 10% to about 80%, and from about 25% to about 75%, by weight of the wetness indicator composition.

### Additional Ingredients

Additional ingredients may include, for example, a stabilizer, a surfactant, a structural adjunct, and/or solvents. When present, such ingredients are typically employed in the composition at levels that are effective at providing the benefits of the ingredient or ingredients, such as, for example, from about 0.001% to about 50%, from about 0.1% to about 40%, or from about 1% to about 35%, by weight of the composition. Solvents may include a liquid, gel or semi-solid material. The solvent may be water, a thixotropic material, paste, an alcohol, ethylene glycol monobutyl ether, mineral oil, esters, silicone fluids, isoparaffins, alkanes like hexane, toluene, xylenes, low molecular weight polyethylene glycols like PEG-400 and PEG-200 and other polyethylene glycols of various molecular weights as marketed by DOW Chemical Incorporated from Midland, Mich., glycerin, glycols, a non-flammable solvent, an adhesive material, or other organic species. Preferred non-aqueous solvents may comprise alcohols, acetates, and combinations thereof. The alcohol solvents are preferably selected from the group consisting of iso-propyl alcohol, n-propyl alcohol, ethanol, methanol, and combinations thereof. Likewise, suitable acetate solvents include, but are not limited to, isopropyl acetate, n-propyl acetate, and combinations thereof.

Other suitable solvents that may be effective include water, aqueous detergent solutions, acidic water solutions, alkaline water solutions, isopropanol, ethanol, methyl-ethyl ketone, acetone, toluene, hexane, ethyl 15 acetate, acetic acid (vinegar), cetyl alcohol (fatty alcohol), dimethicone silicone, isopropyl lanolate, myristate, palmitate, lanolin, lanolin alcohols and oils, octyl dodecanol, oleic acid (olive oil), panthenol (vitamin B-complex derivative), stearic acid and stearyl alcohol, butylene glycol and propy lene glycol, cyclomethicone (volatile silicone), glycerin, aloe, petrolatum, and so forth. Adhesives that may be useful include, for example, those based on alkyds, animal glues, casein glues, cellulose acetates, cellulose acetate butyrates, cellulose nitrates, ethyl celluloses, methyl celluloses, carboxy methyl celluloses, epoxy resins, furane resins, melamine resins, phenolic resins, unsaturated polyesters, polyethylacrylates, poly-methylmethacrylates, polystyrenes, polyvinylacetates, polyvinylalcohols, polyvinyl acetyls, polyvinyl chlorides, polyvinyl acetate chlorides, polyvinylidene copolymers, silicones, starched based vegetable glues, urethanes, acrylonitrile rubbers, polybutene rubbers, chlorinated rubbers, styrene rubbers, and so forth. Waxes such as, for example, polyolefin waxes, bees waxes, and so forth, and gels such as, for example, glycol dimethacrylate, chitosan, polyacrylates, hydroxypropylcellulose, gelatin, and so forth, may also be useful to effect the color change.

Surfactants that are suitable for the present invention may include, for example, ethoxylated alcohols, fatty alcohols, high molecular weight alcohols, ethoxylated sorbitan esters like Tween™ 40 and Span™ 60 and Span™ 65 from Croda, the ethoxylated pareth surfactants like Performathox™ 450 from New Phase Inc. which is owned by Baker Hughes Incorporated, esters, polymers and other natural and synthetic waxes or olefininc materials as known in the art; anionic and cationic surfactants, alkoxylated alkylates such as PEG-20 stearate, end group-capped alkoxylated alcohols, alkoxylated glyceryl and polyglyceryl alkylates such as PEG-30 glyceryl stearate, glyceryl alkylates such as glyceryl stearate, alkoxylated hydrogenated castor oil, alkoxylated lanolin and hydrogenated lanolin, alkoxylated sorbitan alkylates, sugar derived surfactants such as the alkyl glycosides and sugar esters, poloxamers, polysorbates, and sulfo succininc acid alkyl esters. Further examples include nonionic surfactants and amphoteric surfactants and any combination thereof; specific-diethylhexylsodiumsulfosuccinate, available as MONOWET MOE75 from Uniqema, the sodium dioctyl sulfosuccinate line of surfactants like Aerosol™ OT-100 from Cytec Inc. Another example is 4-1-aminoethylphenolpolyoxyethylenefattyethers, polyoxyethylene sorbitan esters, TWEEN, and polyoxyethylene fatty acid esters.

Other suitable surfactants may be neutral block copolymer surfactants, which can be selected from polyoxypropylene-polyoxyethylene block copolymer, poly [poly(ethylene oxide)-block-poly(propylene oxide)]copolymer or propylene glycol-ethylene glycol block copolymer. Suitable neutral polymeric surfactants include TWEEN surfactants, such as TWEEN 20 surfactant, TWEEN 40 surfactant and TWEEN 80 surfactant, and TRITON X-100 surfactant, which are available from Sigma-Aldrich, Incorporated. Other suitable neutral surfactants include polyethylene lauryl ether, polyoxyethylene nonyl phenyl ether, polyoxyethylene oleyl phenyl ether, polyoxyethylene sorbitan monolaurate, polyethylene glycol monostearate, polyethylene glycol sorbitan monolaurate, polyoxyethylenesorbitan monopalmitate, polyoxyethylenesorbitan monostearate, polyoxyethylenesorbitan monooleate, polyoxyethylenesorbitan trioleate, polypropylene glycol sorbitan monolaurate, polyoxypropylenesorbitan monopalmitate, polyoxypropylenesorbitan monostearate, polyoxypropylenesorbitan monooleate, polyoxypropylenesorbitan trioleate, polyalkyne glycol sorbitan monolaurate, polyalkyne glycol sorbitan monopalmitate, polyalkyne glycol sorbitan monostearate, polyalkyne glycol sorbitan monooleate, polyalkyne glycol sorbitan trioleate and mixtures of such neutral surfactants.

The neutral block copolymer based surfactants include PLURONIC series block copolymers, such as PLURONIC P84 or PLURONIC P85 surfactants, which are available from BASF Corporation.

Other suitable neutral block copolymer based surfactants include nonylphenol ethoxylates, linear alkyl alcohol ethoxylate, ethylene oxide-propylene oxide block copolymer, polyoxypropylene-polyoxyethylene block copolymer, polyalkylene oxide block copolymer, polyalkylene oxide block copolymer and propylene glycol-ethylene glycol block copolymer.

It has surprisingly been found that when non-ionic surfactants make up more than 5% by weight of the wetness indicator compositions of the present invention, surfactants with lower HLB values, e.g. lower than 15, are preferable because they help the development of deeper colors from the reaction of the leuco dye(s) with the developer(s), while they also better prevent the premature color formation due e.g. to partial absorption of humidity from air. Therefore when the wetness indicator compositions of the present invention include more than 5% by weight of nonionic surfactant, surfactants with HLB value not higher than 16 are preferable; more preferably the used nonionic surfactants have a HLB value not higher than 13 and most preferably HLB values not higher than 10.

When present, such surfactant or blend of surfactants, is typically employed in the wetness indicator compositions of the present invention at levels that are effective in providing their specific benefits, such as, for example, from about 0.001% to about 90% by weight of the composition, preferably from about 0.1% to about 85%, and more preferably from about 1% to about 80%. It may be desirable to include a stabilizer when the colorant is a pH indicator and when the absorbent article could be stored under conditions of high humidity and high temperature. The inclusion of a stabilizer is also especially important for new diaper designs where materials and/or chemicals are present that could potentially prematurely activate the color change of the colorant within the ink formulation.

In one embodiment of the present invention, the stabilizer is an acidic stabilizer. In another embodiment of the present invention, the stabilizer is a basic stabilizer. The inclusion of a stabilizer, while not wishing to be limited by theory, is believed to play a role in stabilizing the colorant against premature changes caused by exposure to humid environments and/or certain components of the diaper, by maintaining a stable pH, such as a low pH environment with an acidic stabilizer, around the colorant even when the system is exposed to high humidity and/or certain components of the diaper. This maintenance of a stable pH environment keeps the colorant, especially when the colorant is a pH indicator, in its initial dry color state. Desiccants can also stabilize the composition by trapping free water that could prematurely activate the wetness indicator composition. Examples of suitable desiccants include silica gel, bentonite clays, activated alumina, calcium sulfate, copper(II) sulfate, and magnesium sulfate.

One of the key properties of a properly functioning wetness indicator is for it to maintain its dry state color during a variety of storage and packaging conditions while still undergoing a noticeable color change in a reasonable amount of time after being contacted by urine. The colorant should also remain stable to various chemicals and materials that might be present in the diaper. Although alkaline moieties present in the rosins as part of the matrix can aid in preserving the dry state color, additional stabilizer ingredients have been found to be necessary with some new diaper designs where pH components within the diaper can cause the undesirable and premature color change activation of the colorant. To maintain the colorant in its dry state color, alkaliness of suitable strength should be added. Suitable strength is defined by the colorant and pH range where it changes color. The colorant's pKa value is especially important in assessing the characteristics of the chosen stabilizer.

The stabilizer, when present is typically employed in compositions at levels which are effective at stabilizing the colorant, from about 0.001% to about 30%, from about 0.1% to about 15%, and also from about 1% to about 10%, by weight of the composition.

The present invention may include structural adjuncts, such as HLB (hydrophilic lipophilic balance) modifiers, viscosity modifiers, hardening agents, wetting agents, anti-oxidants, anti-leaching aids, and/or colorant solubilizers. Suitable ones may include polymeric thickeners such as block copolymers having polystyrene blocks on both ends of a rubber molecule, the aforementioned copolymers of ethylene and vinyl acetate (EVA), hydrogenated castor oil, polymers, metals salts of fatty acids, silicas and or derivatized silicas, organoclays such as modified and unmodified hectorites and bentonites, modified clays such as modified laponite clays, dibenylidene sorbitol, alkyl galactomannan, aluminium magnesium hydroxide stearate/oil blends and lauroyl glutamic dibutylamide. Hardeining agents may include the aforementioned waxes, C14-22 fatty alcohols, C14-22 fatty acids, C23-60 carboxylic acids, hydrogenated vegetable oils, polymers, sorbitan esters like sorbitan monolaurate, sorbitan monopalmitate, sorbitan monostearate and sorbitan tristearate as trademarked by Croda Incorporated and marketed as SPAN-20, SPAN-40, SPAN-60 and SPAN-65, respectively, and other high molecular weight esters and their derivatives, and mixtures thereof.

The wetting agent can be a surfactant or a mixture of surfactants. The surfactants can be non-ionic surfactants or ionic surfactants. The ionic surfactants can be either positively charged or negatively charged. The examples of non-ionic surfactants include alkyl poly(ethylene oxide) such as copolymers of poly(ethylene oxide) and poly(propylene oxide) (commercially called Poloxamers or Poloxamines), alkyl polyglucosides such as octyl glucoside and decyl maltoside, fatty alcohols such as cetyl alcohol, oleyl alcohol, cocamide MEA and cocamide DEA. The examples of ionic surfactants include anionic (e.g., based on sulfate, sulfonate or carboxylate anions) surfactants such as s(SDS), ammonium lauryl sulfate and other alkyl sulfate salts, Sodium laureth sulfate, also known as sodium lauryl ether sulfate (SLES), Alkyl benzene sulfonate, Soaps, or fatty acid salts like sodium stearate and sodium palmitate and mixtures thereof and Cationic (e.g., based on quaternary ammonium cations) surfactants such as Cetyl trimethylammonium bromide (CTAB) a.k.a. hexadecyl trimethyl ammonium bromide, and other alkyltrimethylammonium salts, Cetylpyridinium chloride (CPC), Polyethoxylated tallow amine (POEA), Benzalkonium chloride (BAC), Benzethonium chloride (BZT); or Zwitterionic (amphoteric) surfactants such as Dodecyl betaine, Dodecyl dimethylamine oxide, Cocamidopropyl betaine, Coco ampho glycinate. Alternatively, the wetting agents may also be hydrophilic molecules. The hydrophilic molecules may be small molecules such as sucrose, glucose and glycerol. The hydrophilicmolecules may also be polymers such as polyethylene glycol and its copolymers.

### Substrate

In one embodiment of the present invention, the wetness indicator composition of the present invention may be on and/or in a substrate. When present on a substrate, the wetness indicator composition will typically be placed on and/or in a substrate where the substrate will be contacted by a liquid, such as water, urine, menses, blood and the like. The substrate may include, but is not limited to, a structural component, such as woven fabrics, nonwoven fabrics, films, sponges, and combinations thereof. The substrate may comprise synthetic and/or natural materials. In one embodiment of the present invention the optional substrate may be an article in its own right, such as, a continuous nonwoven fabric. In another embodiment of the present invention the substrate to which the wetness indicator composition may be applied or otherwise affixed comprises any one, or a combination of, structural components of an absorbent article, including, but not limited to, the backsheet, topsheet, fasteners, absorbent material, etc., or may be a separate element added or applied to the product. In one embodiment of the present invention the wetness indicator composition is applied to the absorbent article as a whole. In some embodiments, the wetness indicator composition is a single layer. Such a single layer may be applied to a substrate or structural component. In some embodiments, the single-layer formulation may be disposed between the backsheet and the absorbent core, in other embodiments, between the topsheet and the absorbent core.

The wetness indicator composition may be coated over a surface of said substrate as either a) a monochromic color scheme alone, bi-chromic, or multiple colors, b) in various shapes and sizes, c) graphics of patterns or alpha numeric symbols and words, or combinations thereof. The color transition may be from being either a) colored to uncolored, b) uncolored to colored, c) colored to different colored, or d) a combination of a) and b) and c).

The following discussion is for convenience of formulation, but is not intended to limit the type of substrate used herein.

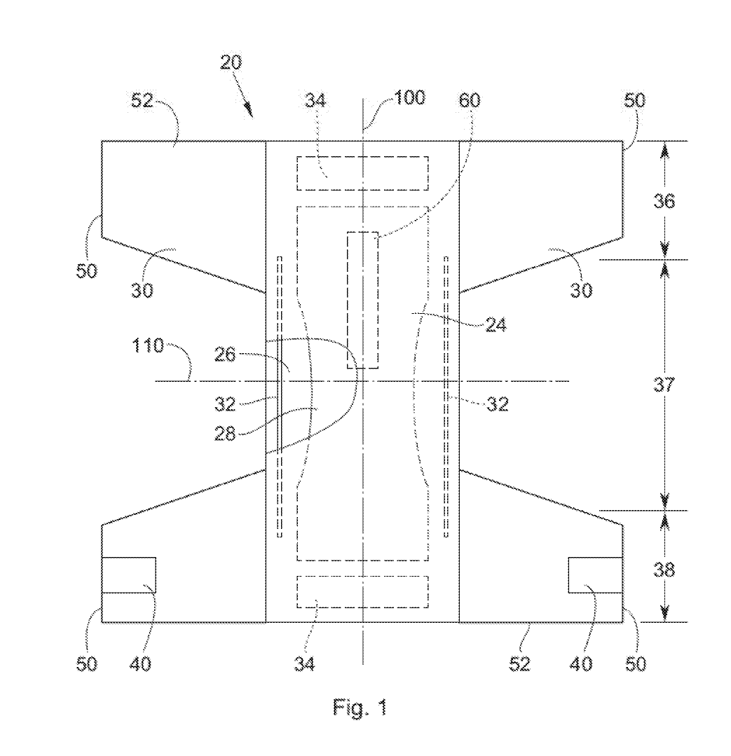

FIG. 1 is a plan view of an absorbent article, in this case a diaper 20, of the present invention in a flat, uncontracted state with portions of the structure being cut away to more clearly show the construction of the diaper. The portion of the diaper 20 that faces a wearer is oriented towards the viewer. As shown in FIG. 1, the diaper 20 comprises a topsheet 24; an outer cover 26; an acquisition layer (not shown), and an absorbent core 28 that is positioned between at least a portion of the topsheet 24 and the backsheet 26. The absorbent article further comprises side panels 30, elasticized leg cuffs 32, elastic waist features 34, and a fastening system generally designated 40. The diaper 20 has a first waist, a second waist region 38 opposed to the first waist region 36, and a crotch region 37 located between the first waist region 36 and the second waist region 38. The periphery of the diaper 20 is defined by the outer edges of the diaper 20 in which longitudinal edges 50 run generally parallel to a longitudinal centerline 100 of the diaper 20 and end edges 52 run between the longitudinal edges 50 generally parallel to a lateral centerline 110 of the diaper 20.

The outermost surface of the backsheet/outer cover 26 forms the garment contacting surface (not shown) of the diaper 20, while the innermost surface of the topsheet 24 forms the body contacting surface (not shown) of the diaper 20. The absorbent articles of the present invention comprise a topsheet 24. In one example, the topsheet 24 is compliant, soft feeling, and non-irritating to the wearer's skin. It can be elastically stretchable in one or two directions. Further, the topsheet is liquid pervious, permitting liquids (e.g., menses, urine, and/or runny feces) to readily penetrate through its thickness. A suitable topsheet can be manufactured from a wide range of materials such as woven and nonwoven materials; apertured or hydroformed thermoplastic films; porous foams; reticulated foams; reticulated thermoplastic films; and thermoplastic scrims. Suitable woven and nonwoven materials may comprise of natural fibers such as wood or cotton fibers; synthetic fibers such as polyester, polypropylene, or polyethylene fibers; or combinations thereof. If the topsheet includes fibers, the fibers may be spunbond, carded, wet-laid, meltblown, hydroentangled, or otherwise processed as is known in the art.

In one embodiment, the backsheet 26 is impervious to fluids (e.g., menses, urine, and/or runny feces) and is manufactured from a thin plastic film, although other flexible liquid impervious materials may also be used. As used herein, the term “flexible” refers to materials which are compliant and will readily conform to the general shape and contours of the human body. The backsheet 26 prevents the exudates absorbed and contained in the absorbent core from wetting articles which contact the absorbent article such as bedsheets, pants, pajamas and undergarments. The backsheet 26 may thus comprise a woven or nonwoven material, polymeric films such as thermoplastic films of polyethylene or polypropylene, and/or composite materials such as a film-coated nonwoven material (i.e., having an inner film layer and an outer nonwoven layer). The backsheet 26 and the topsheet 24 are positioned adjacent a garment surface and a body surface, respectively, of the absorbent core 28.

The articles of the present invention additionally comprise one or more absorbent cores 28. The absorbent core 28 is at least partially disposed between the topsheet and the backsheet and may take on any size or shape that is compatible with the disposable absorbent article. The absorbent core 28 may include any of a wide variety of liquid-absorbent materials commonly used in absorbent articles, such as comminuted wood pulp, which is generally referred to as airfelt. Examples of other suitable absorbent materials for use in the absorbent core include creped cellulose wadding; meltblown polymers including coform; chemically stiffened, modified or cross-linked cellulosic fibers; synthetic fibers such as crimped polyester fibers; peat moss; tissue including tissue wraps and tissue laminates; absorbent foams; absorbent sponges; superabsorbent polymers; absorbent gelling materials (AGM); or any equivalent material or combinations of materials, or mixtures of these. Further useful materials and constructions appropriate for the topsheets, backsheets, outer covers, and absorbent cores described herein may be found in U.S. Ser. No. 14/302,473.

The articles of the present invention may comprise at least one graphic, which refers to images or designs that are constituted by a FIGURE (i.e., a line(s)), a symbol or character, a color difference or transition of at least two colors, or the like. The graphic may have an aesthetic image or design that can provide certain benefits when the absorbent article of the invention is viewed by users or consumers. A variety of graphics can be used in the absorbent articles of the invention.

The article may further comprise at least one wetness indicator 60. A wetness indicator can be located on or against any surface of a component material, including the body contacting surface and the garment contacting surface provided that the wetness indicator 60 remains visible from the exterior of the absorbent article. Non-limiting examples of the component material include the backsheet film/NW, the topsheet, the acquisition layer, the absorbent core, and the barrier leg cuffs. In another embodiment, a wetness indicator 60 is disposed between the absorbent core and the backsheet and in liquid communication with the absorbent core.

### EXAMPLES

The present invention is illustrated by the following examples, which are merely for the purpose of illustration and are not to be regarded as limiting the scope of the invention or the manner in which it can be practiced. Unless specifically indicated otherwise, parts and percentages are given by weight.

### Example 1

| | Wt. % of | |
|---|---|---|
| | wetness indicator | |
| Ingredient | composition | Supplier |
| Crystal Violet Lactone | 6.5 | TCI |
| Salicylic Acid | 1.0 | Sigma-Aldrich |
| Propyl Gallate | 10.5 | Sigma-Aldrich |
| Lauramidopropyl Betaine | 15.5 | Rhodia |
| Polyamide | 11.0 | DuPont |
| Polyethylene imine | 7.7 | BASF |
| Ethylene-vinyl acetate | 47.8 | DuPont |
| Total | 100.0 | |

### Example 2

| | Wt. % of | |
|---|---|---|
| | wetness indicator | |
| Ingredient | composition | Supplier |
| Crystal Violet Lactone | 8.0 | TCI |
| Pergafast 201 | 5.0 | BASF |
| zinc-bis (2-hydroxy-3,5-bis (1 | 9.5 | BOC Sciences |
| phenylethyl) benzoate | | |
| PEG 400 | 11.0 | Dow Chemical |
| PVP-VA | 59.0 | Ashland |
| EVA | 7.5 | Honeywell |
| Total | 100.0 | |

### Examples 1-2 May be Prepared in the Following Manner

Those ingredients that are solids at room temperature are first melted at a temperature which melts the hot melt components and this is typically in the range of 80° C. to 140° C. After melting the ingredient with the highest concentration in the formula, the other ingredients are melted and mixed into the composition. These other ingredients include the binding agents, other hot melt adhesive polymers, waxes, tackifiers, stabilizers, wetting agents like surfactants, colorants like crystal violet lactone, and optional ingredients like the anti-oxidants. Typically, a temperature of around 80° C. to 140° C. would be required to melt these ingredients. After melting to the liquid state on a hot plate, the materials are well mixed using a propeller mixer like an IKA RW-20 (or similar mixer) until the mixture is homogeneous. Heat can be maintained on the composition to make films using a draw-down wire or the composition can be cooled down to room temperature for future use.

### Additional Examples are as Follows

### Example 3

A wetness indicator with the following composition was prepared:

| | Wt. % on the | |
|---|---|---|
| | total weight of the | |
| | wetness indicator | |
| Ingredient | composition | Supplier |
| PVP-VA S-630 | 30.0 | PVP-VA copolymer available |
| copolymer | | from Ashland |
| Span 60 | 42.0 | Non-ionic surfactant available |
| | | from Croda |
| Carbowax 400 | 24.0 | Polyethylene glycol available |
| | | from Dow Chemicals |
| Crystal violet lactone | 1.0 | Leuco dye available from TCI |
| (CVL) | | chemicals |
| 3,5-di-tert-butyl- | 2.5 | Developer available from TCI |
| salicylic acid (TBSA) | | chemicals |
| Irganox 1010 | 0.5 | Antioxidant stabilizer |
| | | available from BASF |

The nonionic surfactant Span 60 plus the Irganox 1010 plus half the weight of Carbowax 400 are heated and melted into a stirred stainless steel mixer at a temperature of 110° C. When the mass is completely molten and homogenous, TBSA is suspended into one fourth of the total quantity of Carbowax 400 and added. Then the PVP-VA copolymer is also slowly added to the molten mass, until it is fully incorporated, melted and homogenously mixed. As a final component the CVL, suspended in the remaining Carbowax 400, is added to the molten composition that is further kept under stirring for about 30 minutes for assuring complete homogeneity.

The resulting wetness indicator coposition, while still in a molten state, is coated on a polyethylene film, typically used e.g. as a backsheet for hygienic absorbent articles, at a basis weight of about 25 grams/m².

The coated surface of the wetness indicator composition appears colorless and remains equally colorless even when stored for 24 hours in conditions of high temperature and humidity, e.g. at 40° C. and 75% relative humidity. However if contacted with a drop of liquid water or of human urine the illustrated colorless wetness indicator composition immediately turns to a dark Sapphire blue color in the wet area.

### Example 4

The wetness indicator composition of example 3 was prepared in the same way but with the modification that 1% by weight of Span 60 was substituted by 1% by weight of zinc oxide added with the rest of Span 60 at the beginning of the preparation. The final wetness indicator composition appears not only distinctly whiter but, when contacted with one drop of water or of human urine, turns immediately to a significantly darker ultramarine blue color. This behavior shows the beneficial effect on the wet color intensity of the wetness indicator compositions of the present invention, given by the addition in the wetness indicator composition of compounds of polyvalent metals and in particular of zinc compounds like zinc oxide.

### Example 5

A wetness indicator with the following composition was prepared:

| | Wt. % on the | |
|---|---|---|
| | total weight of the | |
| | wetness indicator | |
| Ingredient | composition | Supplier |
| AC 5120 | 40.0 | Ethylene Acrylic acid |
| | | copolymer available from |
| | | Honeywell |
| PVP-VA S-630 | 13.0 | PVP-VA copolymer available |
| copolymer | | from Ashland |
| Performathox 420 | 24.0 | Non-ionic surfactant available |
| | | from Baker Hughes |
| Carbowax 400 | 16.0 | Polyethylene glycol available |
| | | from Dow Chemicals |
| 3-(1,2-Dimethyl-3- | 2.0 | Leuco dye available from TCI |
| indolyl)-3-[4-(diethyl- | | chemicals |
| amino)-2-methyl- | | |
| phenyl]phthalide | | |
| 4-4′ di-Hydroxy- | 4.5 | Developer available from TCI |
| benzophenone | | chemicals |
| Irganox 1010 | 0.5 | Antioxidant stabilizer |
| | | available from BASF |

The wetness indicator composition of Example 5 is prepared in a way similar to Example 3 by initially melting in a stirred stainless steel mixer kept at 120° C., the nonionic surfactant Performathox 420 plus the Irganox 1010 and plus half weight of Carbowax 400. About 15 minutes after the complete melting of the above blend, the developer 4-4′ di-Hydroxy-benzophenone is added, after having been suspended into one fourth of the total quantity of Carbowax 400. Then the two polymers AC 5120, first, and PVP-VA, after, are slowly added under continuous stirring until the molten mass is completely homogeneous. Finally the leuco dye is added together with the remaining carbowax.

When coated at a basis weight of about 25 grams/m² on a polyethylene film, the coated wetness indicator composition of Example 5 appears colorless. However when it is contacted with a drop of water or of human urine the wetted area turns quickly to a deep blue turquoise.

### Example 6

A wetness indicator with the following composition was prepared:

| | Wt. % on the | |
|---|---|---|
| | total weight of the | |
| | wetness indicator | |
| Ingredient | composition | Supplier |
| PVP-VA S-630 | 30.0 | PVP-VA copolymer available |
| copolymer | | from Ashland |
| Span 65 | 38.5 | Non-ionic surfactant available |
| | | from Croda |
| Carbowax 400 | 20.0 | Polyethylene glycol available |
| | | from Dow Chemicals |
| Crystal violet lactone | 2.0 | Leuco dye available from TCI |
| (CVL) | | chemicals |
| Pergafast 201 | 8.5 | Developer available from |
| | | BASF |
| Irganox 1010 | 0.5 | Antioxidant stabilizer |
| | | available from BASF |
| Titanium Dioxide | 0.5 | Pigment available from |
| | | Huntsman |

The wetness indicator composition of Example 6 is prepared in a way similar to the previous examples. The non-ionic surfactant and half of the Carbowax 400 are melted at 110° C. in a stirred heated stainless steel mixer and mixed with the Irganox 1010 and the Titanium Dioxide. After about 15 minutes of stirring, Pergafast 201 is added after having been suspended in one fourth of the amount of Carbowax 400. After that the molten mass becomes again homogeneous, the PVP-VA copolymer is slowly added under stirring. Finally the CVL leuco dye is added, mixed with the remaining Carbowax 400. Once coated on a polyethylene film at a basis weight of about 25 g/m², the above wetness indicator composition forms a white coating that, when contacted with water or with human urine, turns in a few seconds to a dark ultramarine blue.

### Example 7

A wetness indicator with the following composition was prepared:

| | Wt. % on the | |
|---|---|---|
| | total weight of the | |
| | wetness indicator | |
| Ingredient | composition | Supplier |
| Polyox WSR N10 | 22.0 | Polyethylene oxide polymer |
| | | available from Dow |
| | | Chemicals |
| Carbowax 8000 | 15.0 | Polyethylene glycol available |
| | | from Dow Chemicals |
| Carbowax 400 | 50.0 | Polyethylene glycol available |
| | | from Dow Chemicals |
| Crystal violet lactone | 2.0 | Leuco dye available from TCI |
| (CVL) | | chemicals |
| Dodecyl Gallate | 10.0 | Developer available from TCI |
| | | Chemicals |
| Irganox 1010 | 0.6 | Antioxidant stabilizer |
| | | available from BASF |
| Tinuvin 770 | 0.2 | UV stabilizer available from |
| | | BASF |
| Tinuvin 928 | 0.2 | UV stabilizer available from |
| | | BASF |

Carbowax 8000, one half of the Carbowax 400, Irganox 1010 and the two Tinuvin stabilizers are heated to 130° C. and melted in a heated stirred stainless steel mixer. When the molten mass is at the desired temperature, the developer Dodecyl Gallate is added together with one fourth of the Carbowax 400. After about 15 minutes of stirring, the Polyox WSR N10 polymer is slowly added until it is homogeneously incorporated into the molten mass. As the final components, the CVL is added together with the rest of the Carbowax 400. The resulting wetness indicator composition is coated on a polyethylene film at a basis weight of about 25 g/m² giving a colorless coating. Said colorless coated wetness indicator composition when contacted with water or with human urine turns immediately, in the wetted areas, to a very dark ultramarine blue.

### Example 8

A wetness indicator with the following composition was prepared:

| | Wt. % on the | |
|---|---|---|
| | totral weight of the | |
| | wetness indicator | |
| Ingredient | composition | Supplier |
| PVP-VA S-630 | 33.0 | PVP-VA copolymer available |
| copolymer | | from Ashland |
| Benzoflex 9-88 | 25.0 | Dipropylene glycol dibenzoate |
| | | plasticizer available from |
| | | Eastman |
| Span 20 | 8.5 | Non-ionic surfactant available |
| | | from Croda |
| Carbowax 4000 | 15.0 | Polyethylene glycol available |
| | | from Dow Chemicals |
| Carbowax 200 | 10.6 | Polyethylene glycol available |
| | | from Dow Chemicals |
| Alpha- | 0.4 | Leuco dye available from TCI |
| naphtholphthalein | | chemicals |
| Sodium stearate | 7.0 | Developer available from |
| | | Aldrich |
| Irganox 1010 | 0.5 | Antioxidant stabilizer |
| | | available from BASF |

Benzoflex 9-88, Carbowax 4000, Span 20 and Irganox 1010 are heated and melted at 110° C. in a heated stirred stainless steel mixer. When the mass is homogeneously melted, Sodium Stearate is suspended in half of the quantity of Carbowax 200 and added to the molten mass. After about 15 minutes of stirring, the PVP-VA copolymer is slowly added and mixed until homogeneously incorporated. Then the leuco dye alpha-naphtholphthalein is added together with the rest of the Carbowax 200. The obtained wetness indicator composition is coated, at a basis weight of about 25 g/m², on a polyethylene film and gives a colorless coating. If some drops of water or of human urine are dripped on the coated wetness indicator composition, the wetted area turns in few seconds to an intense turquoise blue.

### Example 9

A wetness indicator with the following composition was prepared:

| | Wt. % on the | |
|---|---|---|
| | total weight of the | |
| | wetness indicator | |
| Ingredient | composition | Supplier |
| PVP-VA S-630 | 30.0 | PVP-VA copolymer available |
| copolymer | | from Ashland |
| Benzoflex 9-88 | 22.0 | Dipropylene glycol dibenzoate |
| | | plasticizer available from |
| | | Eastman |
| Span 40 | 12.0 | Non-ionic surfactant available |
| | | from Croda |
| Carbowax 4000 | 5.0 | Polyethylene glycol available |
| | | from Dow Chemicals |
| Carbowax 400 | 10.0 | Polyethylene glycol available |
| | | from Dow Chemicals |
| Diethylene glycol | 14.4 | Plasticizer available from TCI |
| | | Chemicals |
| Thymolphthalein | 0.1 | Leuco dye available from TCI |
| | | chemicals |
| Sodium hydroxide | 3.0 | Developer available from |
| | | Aldrich |
| Sodium stearate | 3.0 | Developer available from |
| | | Aldrich |
| Irganox 1010 | 0.5 | Antioxidant stabilizer |
| | | available from BASF |

Benzoflex 9-88, Carbowax 4000, Carbowax 400, Span 40 and Irganox 1010 are heated and melted at 110° C. in a heated stirred stainless steel mixer. Separately from the molten mass inside the stirred stainless steel mixer, sodium hydroxide, used as a 50% by weight water solution, is mixed with sodium stearate and half of the quantity of the diethylene glycol. This blend is heated to 90° C. and kept under stirring at this temperature for about thirty minutes, to be sure that all the water introduced with the sodium hydroxide solution has completely evaporated. At this point the blend of sodium hydroxide, sodium stearate and diethylene glycol is mixed into the stirred stainless steel mixer that contains the molten massat 110° C. When the blend in the stainless steel mixer is homogenously mixed, the copolymer PVP-VA is slowly added until completely melted and dispersed. Then the leuco dye thymolphthalein, blended with the remaining diethylene glycol, is added to the wetness indicator composition. Such wetness indicator composition is coated on a polyethylene film at a basis weight of about 25 g/m², and gives a colorless coating. However when said coating is wetted with water or with human urine, the wet areas turns quickly to a dark ultramarine blue.

### Example 10

A wetness indicator with the following composition was prepared:

| | Wt. % on the | |
|---|---|---|
| | total weight of the | |
| | wetness indicator | |
| Ingredient | composition | Supplier |
| PVP-VA S-630 | 19.0 | PVP-VA copolymer available |
| copolymer | | from Ashland |
| Kraton D 1113 | 15.0 | Styrene-Isoprene-Styrene block |
| | | copolymer available from |
| | | Kraton |
| Benzoflex 9-88 | 25.0 | Dipropylene glycol dibenzoate |
| | | plasticizer available from |
| | | Eastman |
| Diethylene glycol | 19.8 | Plasticizer available from TCI |
| | | Chemicals |
| Foral AX | 10.0 | Tackifier available from |
| | | Eastman |
| Thymolphthalein | 0.2 | Leuco dye available from TCI |
| | | chemicals |
| Sodium hydroxide | 3.0 | Developer available from |
| | | Aldrich |
| Sodium stearate | 3.0 | Developer available from |
| | | Aldrich |
| Span 80 | 3.0 | Non-ionic surfactant available |
| | | from Croda |
| Irganox 1010 | 1.0 | Antioxidant stabilizer |
| | | available from BASF |
| Titanium Dioxide | 1.0 | Pigment available from |
| | | Huntsman |

Benzoflex 9-88, Foral AX, Titanium dioxide, Span 80 and Irganox 1010 are heated and melted at 150° C. In such molten mass Kraton D 1113 is slowly added and mixed until completely homogeneous. Then the temperature of the stirred mixer is cooled down, until the molten mass reaches a temperature of 130° C. Separately from the molten mass, that is kept under stirring inside the mixer, sodium hydroxide, used as a 50% by weight water solution, is mixed with sodium stearate and half of the quantity of the diethylene glycol. This blend is heated to 90° C. and kept under stirring at this temperature for about thirty minutes, to be sure that all the water introduced with the sodium hydroxide solution has completely evaporated. At this point the blend of sodium hydroxide, sodium stearate and diethylene glycol is mixed into the stirred stainless steel mixer that contains the molten mass at 130° C. Then the PVP-VA copolymer is added under continuous stirring until all the mass is homogeneous. The leuco dye thymolphthalein is finally added, mixed with the remaining diethylene glycol. The obtained molten wetness indicator composition is coated on a polyethylene film at a basis weight of about 25 g/m², giving a white coating. When this white coating is wetted with water or with human urine, the wetted area turns from white to a dark steel blue in about one minute.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. An article for baby care or feminine care, comprising a wetness indicator composition comprising:
a) a hot melt binding matrix;
b) a leuco dye;
c) a color developer.

2. The article of claim 1, wherein the leuco dye is selected from the group consisting of phthalide leuco dyes, triarylmethane leuco dyes, fluoran leuco dyes, and combinations thereof.

3. The wetness indicator composition of claim 1, wherein the leuco dye is crystal violet lactone.

4. The wetness indicator composition of claim 1, wherein the leuco dye is 3-(1,2-Dimethyl-3-indolyl)-3-[4-(diethylamino)-2-methylphenyl]phthalide.

5. The wetness indicator composition of claim 2, wherein the leuco dye is from about 0.01% to about 20% by weight of said composition.

6. The wetness indicator composition of claim 1, wherein the color developer functions as a Bronsted or Lewis acid or a strong hydrogen bonding ingredient.

7. The wetness indicator composition of claim 1, wherein the color developer is selected from the group comprising gallic acid, propyl gallate, octyl gallate, dodecyl gallate, salicylic acid, sulfo-salicylic acid, 3,5-di-tert-butylsalicylic acid, acetylsalicylic acid, 2-carboxy-phenyl-salicylate, hydroxy-benzophenones, 4-hydroxy-benzophenone, 4,4′-di-hydroxy-benzophenone, esters of hydroxy-benzoic acids, benzyl-4-hydroxy benzoate, benzoic acid and esters of benzoic acid and its salts, zinc compounds, zinc oxide, zinc chloride, zinc stearate, zinc resinate, zinc salicylate, zinc-bis (2-hydroxy-3,5-bis (1 phenylethyl) benzoate, aluminum sulfate, aluminum double sulfates, silica and silicates, clays, and combinations thereof.

8. The wetness indicator composition of claim 1, wherein the color developer is 3,5-di-tert-butylsalicylic acid (TBSA).

9. The wetness indicator composition of claim 6, wherein the color developer or blend of color developers forms from about 0.1% to about 50% by weight of said composition.

10. The article of claim 1, wherein the hot melt binding matrix comprises at least one binding agent selected from the group consisting of acrylic-based binders, amide based binders, amine based binders, adhesives, hot melt adhesive components, waxes and modified waxes like oxidized waxes, surfactants, rosin esters, rosins and polymerized rosins, modified styrene-acrylic polymers and their salts, polyethylene glycols, polymerized rosins, styrenated terpenes, polyterpene resins, terpene phenolics, quaternary ammonium compounds, quaternary polymers, rubbers, latexes and latex emulsions, cationic clay materials, ethoxylated quaternary ammonium compounds, quaternized silicone compounds, cationic guars, cationic exchange resins, anionic ingredients like anionexchange resins, and combinations thereof.

11. The article of claim 1, wherein the binding matrix comprises a first binding agent and a second binding agent.

12. The article of claim 1, wherein the wetness indicator composition further comprises one or more selected from the group consisting of a stabilizer, a surfactant, a structural adjunct, and combinations thereof.

13. The article of claim 8, wherein said stabilizer is selected from the group consisting of monostearyl phosphate, citrate esters, alcohol ethoxycarboxylates, glycolate esters, lactate esters, fatty acids, ether carboxylic acids, fatty acid methyl esters, sulfate esters, fruit acids, citric acid, malic acid, inorganic acids, sulfuric acid, monoethanolamine, diethanolamine, triethanolamine, dipropyllenetriamine, diiosopropyl amine, 1,3-bis(methylamine)-cyclohexane, 1,3Pentanediamine, sodium hydroxide, magnesium hydroxide, and combinations thereof.

14. The article of claim 8, wherein said surfactant is selected from the group consisting of nonionic surfactants, anionic surfactants, cationic surfactants, amphoteric surfactants, and combinations thereof.

15. The article of claim 8, wherein said structural adjunct is selected from the group consisting of HLB modifiers, viscosity modifiers, hardening agents, anti-leaching aids, anti-oxidants, UV absorbers, UV stabilizers, wetting agents, solvents, and combinations thereof.

16. The article of claim 1, wherein the wetness indicator composition further comprises a permanent colorant.

17. The article of claim 1, wherein the wetness indicator composition further comprises a pH indicator.

18. The article of claim 1, wherein the article comprises a backsheet, a topsheet, and an absorbent core disposed between the backsheet and the topsheet, wherein the wetness indicator is a single layer and disposed between the backsheet and the absorbent core and is affixed to a structural component of the absorbent article.

19. The article of claim 1, wherein the article comprises a backsheet, a topsheet, an absorbent core disposed between the backsheet and the topsheet, wherein the wetness indicator composition is a single layer and disposed between the topsheet and the absorbent core.

20. The article of claim 1, wherein the wetness indicator composition changes from one of colorless, white, or translucent to a visible color.
